SEQUENCE LISTING (34 sequences)

SEQ ID NO: 1 - protein, 447 aa, Dirofilaria immitis
KVEDMLKLVVDKEKKKRIDEYPPVCRKNFNPGNERRKRNDHNLESYFQTYLSWLTDAQKDEIKKMKEEGKSKMDIQKKIFDYFESLTGDKKKKAAEELQQGCLMALSEIIGNEKMLMLKEIKDSGADPEQIEDMLKLVVDKEKKKRIDEYPPVCRKIYAAMNERRKRNDHNLESYFQTYLSWLTDAQKDEIKKMKEEGKSKMDIQKKIFDYFESLTGDKKKKAAEELQGCRMALREIVGEEKWTVLRQMKDSGATPKELSMKVEEMFKDVIDKDKKEKIDEYAPVCRKIFAVIHERRKRNDHNLESYFQTYLSWLTDAQKDEIKKMKEEGKSKMDIQKKIFDYFESLTGDKKKKAAEELQQGCLMALSEIIGNEKMLMLKEIKDSGADPEQIEDMLKLVVDKEKKKRIDEYPPVCRKIYAAMNERRKRNDHNLESYFQTYLSWLTDA

SEQ ID NO: 2 - protein, 586 aa, Dirofilaria immitis
RQMKDSGATPKELSMKVEEMFKDVVDKDKKEKIDEYAPVCHKIFAVIHERRKRNDHNLESYFQTYLSWLTDAQKDEIKKMKEEGKSKMDIQKKIFDYFESLTGDKKKKAAEELQQGCLMALSEIIGNEKMLMLKEIKDSGADPEQIRMKVEDMLKLVVDKEKKKRIDEYAPVCRKIYAAMNERRKRNDHNLESYFQTYLSWLTDAQKDEIKKMEEGGKLKFDTLREITENGKSKADMVVKGFLFYDELFGKAERHVTDLLYDGCRKILKEIIGGDHYEELTEMMDSGADVNDLTVKVDVMLSQITDEEKSEKIKIYRSGCKKIFAKIYYENLLKKLFKTDFKWLTNEQKNEVLRMMVANTSKTDIRVKILHFYDGLSEETKIETVEFFNGVCHDLIVAIFGGETAAELKKLGESSDIANEIRSKMDAIIDKVEDEDRREKAREYGSICQKIFIDYQQEYNKRSLEHYFHTHLKWLSEEQMEEIKKMTTEGKSREEIQSKIFEFFKSASGEAKKSATESLARSCHELFKAIGGENIAHELNVMIRSDIAVNKLEKKIAILIDSMNDESKKTQARVYAIPCMISILFA

SEQ ID NO: 3 - protein, 121 aa, Dirofilaria immitis
YFQTYLSWLTDAQKDEIKKMKEEGKSKMDIQKKIFDYFESLTGDKKKKAAEELQQGCLMALSEIIGNEKMLMLKEIKDSGADPEQIEDMLKLVVDKEKKKRIDEYPPVCRKIYAAMNERRK

SEQ ID NO: 4 - protein, 266 aa, Dirofilaria immitis
SYFQTYLSWLTDAQKDEIKKMKEEGKSKMDIQKKIFDYFESLTGDKKKKAAEELQQGCLMALSEIIGNEKMLMLKEIKDSGADPEQIRMKVEDMLKLVVDKEKKKRIDEYAPVCRKIYAAMNERRKRNDHNLESYFQTYLSWLTDAQKDEIKKMKEEGKSKMDIQKKIFDYFESLTGDKKKKAAEELQQGCLMALSEIIGNEKMLMLKEIKDSGTDPEQIRMKVEDMLKLVVDKEKKKRIDEYAPVCRKIYAAMNERRKRNDHNLE

SEQ ID NO: 5 - protein, 352 aa, Brugia pahangi
RLLSFRHRLDFRSSQGFLRQIVGDEKMAELKQMKESGLGQEELRAKVDEMLEHVTDEAKKQKIHEYGPACRKIYEDRHKRDNHEHSLDDYFRTHLSWLTDAQKDEIRKMKEEGKQKMDMQKKILDYYENLTGDGKKEAGEKLRGGCRELLRQIVGDEKMAELKQMKESGLGQEELRAKVDEMLEHVTDEAKKQKIHEYGPACRKIYEDRHKRDNHEHSLDDYFRTHLSWLTDAQKDEIRKMKEEGKQKMDMQKKILDYYENLTGDGKKEAGEKLRGGCRELLRQIVGDEKMAELKQMKESGLGQEELRAKVDEMLEHVTDEAKKQKIHEYGPACRKIYEDRHKRDNHEHSLG

SEQ ID NO: 6 - protein, 1095 aa, Ascaris suum
TMEHYLKTYLSWLTEEQKEKLKEMKEAGKTKAEIQHEVMHYYDQLHGEEKQQATEKLKVGCKMLLKGIIGEEKVVELRNMKEAGADIQELQQKVEKMLSEVTDEKQKEKVHEYGPACKKIFGATTLQHHRRRRHHFTLESSLDTHLKWLSQEQKDELLKMKKDGKTKKELEAKILHYYDELEGDAKKEATEHLKGGCGEILKHVVGEEKAAELKNLKDSGASKEELKAKVEEALHAVTDEEKKQYIADFGPACKKIYGVHTSRRRRHHFTLESSLDTHLKWLSQEQKDELLKMKKDGKAKKELEAKILHYYDELEGDAKKEATEHLKGGCAEILKHVVGEEKAAELKNLKDSGASKEELKAKVEEALHAVTDEEKKQYIADFGPACKKIYGVHTSRRRRHHFTLESSLDTHLKWLSQEQKDELLKMKKDGKTKKDLQAKILHYYDELEGDAKKEATEHLKDGCREILKHVVGEEKEAELKKLKDSGASKEEVKAKVEEALHAVTDEEKKQYIADFGPACKKIFGAAHTSRRRRHHFTLESSLDTHLKWLSQEQKDELLKMKKDGKAKKELEAKILHYYDELEGDAKKEATEHLKGGCREILKHVVGEEKAAELKNLKDSGASKEELKAKVEEALHAVTDEEKKQYIADFGPACKKIYGVHTSRRRRHHFTLESSLDTHLKWLSQEQKDELLKMKKDGKAKKELEAKILHYYDELEGDAKKEATEHLKGGCREILKHVVGEEKAAELKNLKDSGASKEELKAKVEEALHAVTDEEKKQYIADFGPACKKIYGVHTSRRRRHHFTLESSLDTHLKWLSQEQKDELLKMKKDGKAKKELEAKILHYYDELEGDAKKEATEHLKGGCREILKHVVGEEKAAELKNLKDSGASKEELKAKVEEALHAVTDEEKKQYIADFGPACKKIYGVHTSRRRRHHFTLESSLDTHLKWLSQEQKDELLKMKKDGKAKKELEAKILHYYDELEGDAKKEATEHLKGGCREILKHVVGEEKAAELKNLKDSGASKEELKAKVEEALHAVTDEEKKQYIADFGPACKKIYGVHTSRRRRHHFTLESSLDTHLKWLSQEQKDELLKMKKD

SEQ ID NO: 7 - protein, 404 aa, Ostertagia ostertagi
HSLEDAMGKYLTWLTDDQKEEVKSLYTDEGRGAVYDKIMEYFDEATGDRKEKAAKELKGACKHYVKDLIGEKNGEMIKEMKENGASNDAIATKVEELIEAIADDKKKAQALRASANCRKIYGVARRFRRDHHEHNLEEAMEKYLTWLNDDQKEEVKKLYGAGDKQAMYKKVMEIYDSVSGDVKEKATVELKAACRHYVKDSIGEENAEKLKEMKESGATPEAIAAKVEEFIAAITDEKKKAQAERAAVACKKIYGVARRLKREHHEHNLEEAMEKYLTWLNDEQKEEVKKIYGTGDRIAVETKVLQMFENASGDVKEKASVQLRAACKHYIKEYIGDENVAKIKEMKDSGASNEAMSAKIDEFIAAIPEKERKEKAERVAASCKKVYGVKSRMRRYPARSTRST

SEQ ID NO: 8 - protein, 587 aa, Loa Loa
DHHEHNLDEYFRTHLSWLTDIQKDEIRKMKEEGKPKADMQKKIFDYYESLTGDEKKEASEKLREGCRALLKQIVGDEKMAELKQMKDSGDGYEELIAKVDHMLEHVTDEPKKEKITEYGPACRKIYGDRHKRDHHEHNLDEYFRTHLSWLTDIQKDEIRKMKEEGKPKADMQKKIFDYYESLTGDEKKEASEKLREGCRALLKQIVGDEKMAELKQMKDSGVGIEELIAKVDHMLEHVTDEPKKEKIQEYGPACHKIYGPVMRNKRNQANNGTKHCSSPYLQWLTDEEKGEIRKIIGGNKSRADILKAIFFYYQILPGKEKKNAGERLRLSCEEIVRSLVYENRLSELEALENDSSTMSEMMKMLSAATDRSKFAQIKAYQTACNRIFDLRQLVRRKREHKGNSIDNYLEKNLKWLSVEQREELREMKKNGKSRADMIAKMFHYYEELFGEAKQHVTERLYDGCRQILKDVVGGDRYNELAKMKDSGANMNDLKAKADAMLNEIIDEEKKEKIKIYGSGCMRIFTRMGHKHSLEEHFKTDLKWLTKEQKDELLKMKEENKSEADIREKVLHFYESLNEEAKKETAEF

SEQ ID NO: 9 - protein, 1557 aa, Dictyocaulus viviparus
MKSTSFITLLLLSYFIVEAHSSIFHWDDERLFKHDDTHSWLTDVQKAELETLKHQPIQLRDKTLEFYNQLPTNEKAIWDKFYTKYCVVWLKEVASDEEIGKLKELESEKNKEALLTSIYSFKDRLDEVDQRKVELWKETCDEYVTKGLSRKRRDSNKNFEEFIYWMTDEQKQSMNDMKTAGKSFNEIHKEGRKYFKALTIDKQSSLKEQFKDKCKKYFMQIANSDEVEKIKSLNDDEIRHVVKNAVARLNGEDKEFAVKMETLCEDVLAFKARKNDIDDKINRRLSWMTDEQKQVVKQLYADGRSQADIRAKIFEFLSSIDGPAGVAAKAQIQKECYKWMEEVATAEEIAALHELHEIDHDGCRRKVREFIGRLPEDRKLEVEKDLPFCEKIWYRDHGDHNSHKHGAHHHHRHLAVRRRRHLYAIEKFLDWLKPEQKHELEKIENSGAHFDDVIAEVKKFYGLLPEEKKIELKAKFKSQCYDWVKEVATSEEMNDIMKMHESKNHSDLMKRLTELENRLTEDQKHTIEHVREVCLGLWEVQNTNKQHKQSLEEAMDAYLSWMTDEDKEKVKAIYETSNRQTFYDEILKIMESSEDEVKAKATEKLEAACKHYGTNILGEENVDIIREMKKNGATFEEISNRVDELIEGITDSDRKEKAYRMSKLCKKIYSLGHSKQLQQYDFENVLQKYLTWLDDSQKNELRTMSDNKEKIYKKIIDYFDGTIGEVKEKAVEELQLACNHYIKSIVGEEKAMEIKQLKEEGKSSEEIAKKVEDVINQISDESIRSRADEALLVCKRIFGIVKRLRRDNSEIHSLEEAMERYLTWLSDDQKIVIKSIYDVNDRKVLYEKIMEFFDDAIGETKQKAAKELKDACKHYVKDLIGEENGNLLREMKENGASNEAIATKVEEMIEAITDETKRAQAMRASTSCRKVYGVVQRFRRDHHHEHNLDEALEKHFTWLNEEQKSQLKTIYESEDREALHKKVWEFFEAGAGLRASNASKKIYGVAKRFRRDHHHEHNLDEALEKYLTWLNEEQKSQMKTIYESGDREALYKKVLEFFEAATGEVKEKAAVELKSACRHYIKDYIGDEKAEKIKEMKESGVSTEEISKKVDEFIAMITDDEKKAKALRASSACKKIYGVAKRFRRDHHHEHNLEEALEKYLTWLNEEQKSQMKTIYESGDREALYKKVLEFFEAATGEVKEKAAVELKSACRHYIKDYIGDEKAEKIKEMKESGVSTEEISKKVDEFIAMITDDEKKAKALRASNACKKIYGVAKRLRRDHHHEHNLEEAMGKYLSWMSDEQQAQVKKIYGTGDRLATYNKVMELFESVPSDEKEKATSQLKAACRHYIKDFIGKDNLAVIKEMKESGATNEAIGEKIDEFIAGLDDEQKKAQAQRAASACKKIYGVKSRKRREHYEIDVDEAISKYLTWLNEEQKAEIKQLKEKDEKQTIGKKIMEFFELTSGDDKEKAREQLKAACKHYVKMYVGEEKAAELKKLKDSGISLEEMSKKVTETIETIEDEAVRAKARRIHSYCQRIFGITKARRHLAMKHHRFYDD

SEQ ID NO: 10 - protein, 133 aa, Ochotona princeps
NHHNLESYFRTHLSWLTDAQKDEIKKMKEEGNRKMDIQKKIFDYFESLTGDKKKKAAEELQEGCRMAMREIVGEEKWTVLRPMKDSGPTPKELSMKVEEMFKDVFDKDKKVKIDEYAPVCRKILPVIHERRKR

SEQ ID NO: 11 - protein, 235 aa, Wuchereria bancrofti
  MISC_FEATURE (146)..(146): any amino acid
  MISC_FEATURE (167)..(167): any amino acid
  MISC_FEATURE (175)..(175): any amino acid
  MISC_FEATURE (176)..(176): any amino acid
  MISC_FEATURE (180)..(180): any amino acid
  MISC_FEATURE (190)..(190): any amnino acid
  MISC_FEATURE (191)..(191): any amino acid
  MISC_FEATURE (196)..(196): any amino acid
  MISC_FEATURE (197)..(197): any amino acid
  MISC_FEATURE (198)..(198): any amino acid
  MISC_FEATURE (201)..(201): any amino acid
  MISC_FEATURE (204)..(204): any amino acid
  MISC_FEATURE (207)..(207): any amino acid
  MISC_FEATURE (211)..(211): any amino acid
  MISC_FEATURE (212)..(212): any amino acid
  MISC_FEATURE (213)..(213): any amino acid
  MISC_FEATURE (219)..(219): any amino acid
  MISC_FEATURE (220)..(220): any amino acid
  MISC_FEATURE (225)..(225): any amino acid
  MISC_FEATURE (228)..(228): any amino acid
  MISC_FEATURE (229)..(229): any amino acid
  MISC_FEATURE (231)..(231): any amino acid
IYEDRYKRDNHEHSLDDYFRTHLSWLTDAQKDEIRKMKEEGKPKIDMQKKIFDYYENLTGDGKKEAGEKLRGGCRELLRQIVGDEKMAELKQMKESGLGQEELIAKVDEMLGHITDEAKKQKIHEYGPSCRKIYEDRFLRDNHEHXLDDYFRTHLSWLTDAQIDDIXKMKEEGSXXNRYXRKRSLITTTXXPVTEXXXPVXNFXGRXPLNXXXKLFGDXXMAEFXPNXXIXIPVR

SEQ ID NO: 12 - protein, 132 aa, Brugia malayi
EGKQKMDMQKKILDYYENLTGDGKKEAGEKLRGGCRELLRQIVGDEKMAELKQMKESGLGQEELRAKVDEMLEHVTDEAKKQKIHEYGPACRKIYEDRHKRDNHEHSLDDYFRTHLSWLTDAQKDEIRKMKE

SEQ ID NO: 13 - protein, 133 aa, Setaria cervi
DEHTLESYFQTHLSWLTDIQKDEIRKMKEEGKSKAEIQKTVFHYYDGLTGDKKKEAVEKLRGGCNELLKQIVGEEKVAELKRMKESGMDFEQIKAKVESILDHVTDETQKQKVQEYGAACRKVYAETDSRQKR

SEQ ID NO: 14 - protein, 352 aa, Brugia pahangi; identical to SEQ ID NO: 5

SEQ ID NO: 15 - protein, 136 aa, Litomosoides carinii
YHHEHSLEEYFQTHLSWLTDAEKDEIRKMKQEGKPKAEIQQKIFGFTYYENMTGDAKKEAGEKLRRGCRQLLKQIVGEEKMSELKQMKDSGADLKTLAAKVDEMLEFTHVTDEAKRKTIQEYGSACRKIYEERHKR

SEQ ID NO: 16 - protein, 267 aa, Ascaris lumbricoides
HTMEHYLKTYLSWLTEEQKEKLKEMKEAGKTKAEIQHEVMHYYDQLHGEEKQQATEKLKVGCKMLLKGIIGEEKVVELRNMKEAGADIQELQQKVEKMLSEVTDEKQKEKVHEYGPACKKIFGATTLQHHRRRRHHFTLESSLDTHLKWLSQEQKDELLKMKKDGKTKKELEAKILHYYDELEGDAKKEATEQLKGGCREILKHVVGEEKAAELKNLKDSGASKEELKAKVEEALHAVTDEEKKQYIADFGPACKKIYGVHTSRRRR

SEQ ID NO: 17 - protein, 131 aa, Acanthocheilonema viteae
EHSPPRYFRPHLSWLTDAQKDEVLKMEVENKARADIQGKILHFYEDLNEEAKKEAAEFLNGACYDITVHVFGDEKAEELKKVRESTGVSDEIRRKMDGMIDEIEDEDQKTKAQEYGPICQNIFLHYQRKHR

SEQ ID NO: 18 - protein, 1557 aa, Dictyocaulus viviparus; identical to SEQ ID NO: 9

SEQ ID NO: 19 - protein, 404 aa, Ostertagia ostertagi; identical to SEQ ID NO: 7

SEQ ID NO: 20 - DNA, 1342 bp, Dirofilaria immitis
aaagtcgaagatatgttgaaacttgtcgttgacaaagaaaagaagaaaagaattgatgaatatcctcctgtatgccgtaaaaattttaatcccggcaatgaacggcgtaagcggaatgatcataatttagaaagctattttcagacgtatctgagctggctcacagatgctcaaaaagatgaaattaaaaaaatgaaagaagaaggaaaatcgaaaatggatattcaaaaaaaaatttttgattatttcgaaagtttgacaggtgataaaaagaaaaaggctgcagaagaacttcaacaaggttgcttaatggctctcagtgaaatcattggtaatgaaaagatgcttatgttgaaagagattaaagattcaggcgctgatccagaacaaatcgaagatatgttgaaacttgtcgttgacaaagaaaagaagaaaagaattgatgaatatcctcctgtatgccgtaaaatttatgcggcaatgaatgaacggcgtaagcggaatgatcataatttagaaagctattttcagacgtatctgagctggctcacagatgctcaaaaagatgaaattaaaaaaatgaaagaagaaggaaaatcgaaaatggatattcaaaaaaaaatttttgattatttcgaaagtttgacaggtgataaaaagaaaaaggctgcagaagaacttcaaggctgcagaatggctctgagagaaattgttggtgaagagaagtggactgtattgaggcaaatgaaggattcaggcgcaactccaaaggaactaagcatgaaagttgaagagatgttcaaagatgtcattgacaaagataaaaaggaaaaaattgatgaatatgctcctgtatgccgtaaaatctttgcggtgatacatgaaaggcgtaagcggaatgatcataatttagaaagctattttcaaacgtatctgagctggctcacggatgctcaaaaagatgaaattaaaaaaatgaaagaagaaggaaaatcgaaaatggatattcaaaaaaaaatttttgattatttcgaaagtttgacaggtgataaaaagaaaaaggctgcagaagaacttcaacaaggttgcttaatggctctcagtgaaatcattggtaatgaaaagatgcttatgttgaaagagattaaagattcaggcgctgatccagaacaaatcgaagatatgttgaaacttgtcgttgacaaagaaaagaagaaaagaattgatgaatatcctcctgtatgccgtaaaatttatgcggcaatgaatgaacggcgtaagcggaatgatcataatttagaaagctattttcagacgtatctgagctggctcacagatgctc

SEQ ID NO: 21 - DNA, 399 bp, Onchocerca cervicalis
aatcatcataatttagaaagctattttcgaacgcatctaagctggctcacggatgcccaaaaagatgaaattaaaaaaatgaaagaagaaggaaatcgaaaaatggatattcaaaaaaaaatttttgattatttcgaaagtttgacaggtgataaaaagaaaaaggctgcagaagaacttcaagaaggctgcagaatggctatgagagaaattgttggtgaagagaagtggactgtattgaggccaatgaaggattcaggcccaactccaaaggaactaagcatgaaagttgaagagatgttcaaagatgtgttcgacaaagataaaaaagtaaaaattgatgaatatgctcctgtatgccgtaaaatcttgccggtgatacatgaaaggcgtaagcgg

SEQ ID NO: 22 - DNA, 399 bp, Setaria cervi
gacgagcataccttggaaagctatttccaaacgcatctgagctggcttactgatattcaaaaagatgaaattcggaaaatgaaagaggaagggaagtcgaaagcagagatccagaaaacagtattccattattacgatggcttaacgggtgacaaaaagaaggaagcggtggagaaacttcgtggcggatgcaatgaattactgaagcaaattgttggtgaggaaaaagttgctgaattgaaaagaatgaaagaatcaggtatggattttgaacagataaaagccaaagtggagagcatattggatcatgtgactgatgaaactcagaagcagaaagtacaagagtatggtgctgcatgccgtaaagtttatgcagagaccgacagtcgacagaaacgc

SEQ ID NO: 23 - DNA, 1055 bp, Brugia pahangi
cggcttctttctttccgtcaccggttagattttcgtagtagtcaaggatttttgaggcaaattgttggtgatgaaaagatggctgaattaaaacagatgaaagaatcaggactcggtcaggaagaactgagagctaaagtagatgaaatgctggaacatgttactgatgaagccaagaagcaaaaaattcatgaatatggccctgcatgccgtaaaatctatgaggatcgacataaacgagataaccatgagcatagtttagatgactattttcggacgcatctaagttggcttacggatgcccaaaaggatgaaatcaggaaaatgaaagaggaaggtaaacaaaaaatggatatgcagaaaaaaatccttgactactacgaaaatctaaccggtgacggaaagaaagaagccggtgagaaactccgtggaggttgtcgtgaattattgaggcaaattgttggtgatgaaaagatggctgaattaaaacagatgaaagaatcaggactcggtcaggaagaactgagagctaaagtagatgaaatgctggaacatgttactgatgaagccaagaagcaaaaaattcatgaatatggccctgcatgccgtaaaatctatgaggatcgacataaacgagataaccatgagcatagtttagatgactattttcggacgcatctaagttggcttacggatgcccaaaaggatgaaatcaggaaaatgaaagaggaaggtaaacaaaaaatggatatgcagaaaaaaatccttgactactacgaaaatctaaccggtgacggaaagaaagaagccggtgagaaactccgtggaggttgtcgtgaattattgaggcaaattgttggtgatgaaaagatggctgaattaaaacagatgaaagaatcaggactcggtcaggaagaactgagagctaaagtagatgaaatgctggaacatgttactgatgaagccaagaagcaaaaaattcatgaatatggccctgcatgccgtaaaatctatgaggatcgacataaacgagataaccatgagcatagtttagg

SEQ ID NO: 24 - DNA, 1764 bp, Loa Loa
gatcatcatgaacacaatttggatgagtacttccgaacgcatctaagctggctcacggatatccagaaagatgaaattaggaaaatgaaggaagaaggaaaaccgaaagcggatatgcaaaagaaaatttttgattattacgaaagtctaaccggtgatgaaaagaaggaagctagtgagaagcttcgagaaggctgccgcgcattgctgaagcaaattgttggtgatgagaagatggctgaactaaaacagatgaaagattcgggcgatggatatgaggaactgatagccaaagtggatcatatgttggaacatgttactgatgagccaaaaaaggaaaaaattacggaatatggtcctgcatgccgtaaaatctatggggatcgacataagcgggatcatcatgaacacaatttggatgagtacttccgaacgcatctaagctggctcacggatatccagaaagatgaaattaggaaaatgaaggaagaaggaaaaccgaaagcggatatgcaaaagaaaatttttgattattacgaaagtctaaccggtgatgaaaagaaggaagctagtgagaagcttcgagaaggctgccgcgcattgctgaagcaaattgttggtgatgagaagatggctgaactaaaacagatgaaagattcgggcgtaggtatagaggaactgatagccaaagtggatcatatgttggaacatgttactgatgagccaaaaaaggaaaaaattcaggaatatggtcctgcatgccataaaatctacggaacacctgtgatgcggaataagcggaatcaggcaaataatggcacaaaacactgcagcagtccttacttgcaatggcttactgacgaagaaaaaggcgaaattcgaaaaattatagggggaaacaaatcaagagccgatatactgaaagctatctttttttactatcaaattcttcctggaaaggagaagaaaaatgcgggagagcgcttgaggttaagttgcgaagaaatcgttcgcagtttggtctacgaaaatcgattaagtgaattagaagctttggaaaatgatagttctacaatgagcgagatgatgaaaatgttgtcagctgcaacggatagatcaaagtttgcacaaattaaggcatatcaaaccgcttgtaacagaatcttcgatttgagacagctggtgagaagaaaacgcgaacacaaaggtaattcaatcgataattatcttgagaagaatttaaagtggctttcggtggagcagagggaggaattgagggaaatgaagaagaatggtaaatcgagggctgatatgattgctaaaatgtttcattattacgaagaattatttggagaagcaaagcagcatgtcactgaacggttatacgacggttgtcgacaaattttaaaagatgttgttggtggagatcgctacaatgaattggcaaaaatgaaggattcgggtgcaaatatgaatgatttaaaagcgaaagctgatgcaatgttgaatgaaataatagacgaagaaaagaaggaaaaaattaaaatttacggatctggatgcatgagaatttttacaaggatgggtcataaacattcattggaggaacatttcaagacagatctgaaatggctcacaaaggaacaaaaggatgagctactaaagatgaaggaggaaaacaaatcggaagcagatattcgagaaaaagtattgcacttctacgaaagtctaaatgaagaagctaaaaaagaaacggctgaattc

SEQ ID NO: 25 - DNA, 707 bp, Wuchereria bancrofti
  misc_feature (499)..(499): any nucleotide base
  misc_feature (523)..(523): any nucleotide base
  misc_feature (527)..(527): any nucleotide base
  misc_feature (539)..(539): any nucleotide base
  misc_feature (567)..(567): any nucleotide base
  misc_feature (568)..(568): any nucleotide base
  misc_feature (573)..(573): any nucleotide base
  misc_feature (586)..(586): any nucleotide base
  misc_feature (587)..(587): any nucleotide base
  misc_feature (590)..(590): any nucleotide base
  misc_feature (594)..(594): any nucleotide base
  misc_feature (602)..(602): any nucleotide base
  misc_feature (611)..(611): any nucleotide base
  misc_feature (621)..(621): any nucleotide base
  misc_feature (623)..(623): any nucleotide base
  misc_feature (632)..(632): any nucleotide base
  misc_feature (635)..(635): any nucleotide base
  misc_feature (637)..(637): any nucleotide base
  misc_feature (655)..(655): any nucleotide base
  misc_feature (660)..(660): any nucleotide base
  misc_feature (673)..(673): any nucleotide base
  misc_feature (674)..(674): any nucleotide base
  misc_feature (678)..(678): any nucleotide base
  misc_feature (682)..(682): any nucleotide base
  misc_feature (686)..(686): any nucleotide base
  misc_feature (692)..(692): any nucleotide base
atctatgaggatcgatataagcgagataaccatgagcatagtctagatgattatttccggacgcatctaagttggcttacggatgcccagaaagatgaaatcaggaaaatgaaagaggaaggtaaaccaaaaatagatatgcagaaaaagatctttgattactacgaaaatctaaccggtgacggaaagaaagaagccggtgagaaacttcggggaggttgccgtgaattattgagacaaattgttggtgatgagaagatggctgaattaaaacagatgaaagaatcaggactcggtcaggaagaactgatagctaaagtcgatgaaatgctgggacatattactgatgaagccaagaagcaaaaaattcatgaatatggaccttcatgccgtaaaatctatgaggatcgatttctgcgagataaccatgagcatmgtttagatgattatttccggacgcatctaagttggcttacggatgcccagatagatgacatcnagaaaatgaaagaggaaggttcanccanaaatagatatgnccgaaaacgatctttgattactacgacnntcttnccggtgacggaannaangaanccggtganaaacttccngggaaggttncccttaaattnttnanacaaattgtttggtgatnataanatggctgaatttnnaccnaatnaaanaatccngattccggtccggaa

SEQ ID NO: 26 - DNA, 396 bp, Litomosoides carinii
taccatcatgaacacagtttggaggagtacttccaaacacatctgagttggcttacagatgctgagaaagatgaaatcagaaaaatgaaacaagaaggaaaaccaaaagcagaaattcaacagaagatttttggttattacgaaaatatgaccggcgacgcaaagaaggaagcgggtgagaaacttcgtagaggatgtcgccaactgttgaagcaaatcgttggtgaggaaaagatgtctgagttaaaacagatgaaagattcaggagccgatctaaaaacgcttgcagcgaaagtggacgaaatgcttgagcatgtcaccgacgaagcaaagaggaaaacgatccaggaatatggttctgcttgtcgcaaaatctacgaagagcgacacaagaga

SEQ ID NO: 27 - DNA, 1284 bp, Dirofilaria immitis
cggaatgatcataatttagaaagctattttcagacgtatctgagctggctcacagatgctcaaaaagatgaaattaaaaaaatgaaagaagaaggaaaatcgaaaatggatattcaaaaaaaaatttttgattatttcgaaagtttgacaggtgataaaaagaaaaaggctgcagaagaacttcaacaaggttgcttaatggctctcagtgaaatcattggtaatgaaaagatgcttatgttgaaagagattaaagattcaggcgctgatccagaacaaatcagaatgaaagtcgaagatatgttgaaacttgtcgttgacaaagaaaagaagaaaagaattgatgaatatgctcctgtatgccgtaaaatttatgcggcaatgaatgaacggcgtaagcggaatgatcataatttagaaagctattttcagacgtatctgagctggctcacagatgctcaaaaagatgaaattaaaaaaatgaaagaagaaggaaaatcgaaaatggatattcaaaaaaaaatttttgattatttcgaaagtttgacaggtgataaaaagaaaaaggctgcagaagaacttcaagaaggctgcagaatggctctgagagaaattgttggtgaagagaagtggactgtattgaggcaaatgaaggattcaggcgcaactccaaaggaactaagcatgaaagttgaagagatgttcaaagatgtcgttgacaaagataaaaaggaaaaaattgatgaatatgctcctgtatgccgtaaaatctttgcggtgatacatgaaaggcgtaagcggaatgatcataatttagaaagctattttcaaacgtatctgagctggctcacagatgctcaaaaagatgaaattaaaaaaatgaaagaagaaggaaaatcgaaaatggatattcaaaaaaaaatttttgattatttcgaaagtttgacaggtgataaaaagaaaaaggctgcagaagaacttcaagaaggctgcagaatggctctgagagaaattgttggtgaagagaagtggactgtattgaggcaaatgaaggattcaggcgcaactccaaaggaactaagcatgaaagttgaagagatgttcaaagatgtcgttgacaaagataaaaaggaaaaaattgatgaatatgctcctgtatgccgtaaaatctttgcggtgatacatgaaaggcgtaagcggaatgatcataatttagaaagctattttcaaacgtatctgagctggctcacggatgctcaaaaagatgaaattaaaaaaaaaaaa

SEQ ID NO: 28 - DNA, 788 bp, Dirofilaria immitis
agctattttcagacgtatctgagctggctcacagatgctcaaaaagatgaaattaaaaaaatgaaagaagaaggaaaatcgaaaatggatattcaaaaaaaaatttttgattatttcgaaagtttgacaggtgataaaaagaaaaaggctgcagaagaacttcaacaaggttgcttaatggctctcagtgaaatcattggtaatgaaaagatgcttatgttgaaagagattaaagattcaggcgctgatccagaacaaatcagaatgaaagtcgaagatatgttgaaacttgtcgttgacaaagaaaagaagaaaagaattgatgaatatgctcctgtatgccgtaaaatttatgcggcaatgaatgaacggcgtaagcggaatgatcataatttagaaagctattttcagacgtatctgcagatgctcaaaaagatgaaattaaaaaaatgaaagaagaaggaaaatcgaaaatggatattcaaaaaaaaatttttgattatttcgaaagtttgacaggtgataaaaagaaaaaggctgcagaagaacttcaacaaggttgcttaatggctctcagtgaaatcattggtaatgaaaagatgcttatgttgaaagagattaaagattcaggcactgatccagaacaaatcagaatgaaagtcgaagatatgttgaaacttgtcgttgacaaagaaaagaagaaaagaattgatgaatatgctcctgtatgccgtaaaatttatgcggcaatgaatgaacggcgtaagcggaatgatcataatttagaa

SEQ ID NO: 29 - DNA, 1905 bp, Dirofilaria immitis
cggcaaatgaaggattcaggcgcaactccaaaggaactaagcatgaaagttgaagagatgttcaaagatgtcgttgacaaagataaaaaggaaaaaattgatgaatatgctcctgtatgccataaaatctttgcggtgatacatgaaaggcgtaagcggaatgatcataatttagaaagctattttcagacgtatctgagctggctcacagatgctcaaaaagatgaaattaaaaaaatgaaagaagaaggaaaatcgaaaatggatattcaaaaaaaaatttttgattatttcgaaagtttgacaggtgataaaaagaaaaaggctgcagaagaacttcaacaaggttgcttaatggctctcagtgaaatcattggtaatgaaaagatgcttatgttgaaagagattaaagattcaggcgctgatccagaacaaatcagaatgaaagtcgaagatatgttgaaacttgtcgttgacaaagaaaagaagaaaagaattgatgaatatgctcctgtatgccgtaaaatttatgcggcaatgaatgaacggcgtaagcggaatgatcataatttagaaagctattttcagacgtatctgagctggctcacagatgctcaaaaagatgaaattaaaaaaatggaagaaggaggaaaattgaaatttgacacactgagggaaattacggagaatggaaaatcgaaagctgatatggttgttaagggttttcttttttatgatgaattatttggcaaagcagaacggcatgtcactgatttgttatatgatggttgccgaaaaattttaaaagagattattggtggggatcactatgaagaattgacagaaatgatggactcgggtgcagatgtaaacgatttaacagtaaaagttgatgtaatgttgagtcaaataacagacgaggagaagagcgaaaaaattaaaatttacaggtccggttgcaaaaaaatatttgcaaaaatatattatgaaaatttattaaagaagctcttcaaaacggattttaaatggcttacgaatgaacagaagaatgaagtgttaaggatgatggtggcgaacacatcgaaaacagatattcgagtaaaaatattacatttctatgatggtttaagtgaagaaactaaaatagaaacagttgaattctttaatggtgtatgtcacgatttgattgtggcaatttttggtggtgaaacagcagcagaattgaagaaattaggagaatcaagtgatattgctaatgaaattagaagcaagatggatgcaataattgataaggttgaggatgaagacaggagagaaaaagcaagagaatatggctcaatttgtcaaaaaatcttcattgattaccaacaagaatataataaacgttcgctagagcattactttcacacgcatctgaagtggctttctgaagaacagatggaagaaatcaagaaaatgacaactgaaggaaaaagccgtgaagaaattcaatccaaaattttcgaattttttaaaagtgcaagcggtgaagcgaagaaatctgctacagaatcattagcgcgaagttgtcacgaattatttaaagcaattggcggtgaaaatatagctcatgagttgaatgttatgatacgcagtgatatagctgtcaataagctcgaaaagaaaattgctatattaatcgattccatgaatgatgaatcaaagaagactcaagctagagtttacgcaataccatgcatgatctccatactattcgcttgaataagcgaattcggggaaattatttcacattatttaacacaggtttactaatctaacatatagcagttatttaaaagtaatttttctaaacacttaaaaatgtttctttacattaagtgtattcaataaattgcagattcttca

SEQ ID NO: 30 - DNA, 801 bp, Ascaris lumbricoides
catacaatggaacactatctcaaaacctatctgagctggctgacagaagaacaaaaagaaaagctgaaagaaatgaaagaggcaggcaaaacgaaggcagagatccaacatgaagtgatgcactactacgatcaactgcatggtgaagaaaaacaacaagcaacagaaaagctcaaagtgggctgcaaaatgctcctgaaaggaatcatcggcgaggaaaaggtagttgagttgaggaacatgaaggaagcaggagcagacattcaagaacttcaacaaaaggttgagaagatgctttccgaggtcacagacgaaaagcagaaagaaaaagtccacgagtatggacccgcatgcaaaaagatcttcggtgcgacaacactgcaacatcatcgacgaaggaggcatcatttcacccttgaaagcagtctagatacccatctgaaatggctcagtcaggaacagaaagatgaattgttgaaaatgaagaaagatggaaagacaaagaaagagctcgaggcgaaaattcttcattactatgatgaactcgaaggagatgctaaaaaagaggcaactgagcaattgaaaggcggatgccgcgaaattcttaagcatgttgttggggaagagaaggcagcagagctgaagaacctcaaagactcgggagcaagcaaagaagaactcaaagccaaagtcgaagaggcgcttcatgcagtgaccgacgaggagaagaagcaatacattgccgattttggaccagcatgcaagaaaatctatggtgtacatacttcgcgacgaaggagg

SEQ ID NO: 31 - DNA, 3287 bp, Ascaris suum
atacaatggaacactatctcaaaacctatctgagctggctgacagaagaacaaaaagaaaagctgaaagaaatgaaagaggcaggcaaaacgaaggcagagatccaacatgaagtgatgcactactacgatcaactgcatggtgaagaaaaacaacaagcaacagaaaagctcaaagtgggctgcaaaatgctcctgaaaggaatcatcggcgaggaaaaggtagttgagctgaggaacatgaaggaagcaggagcagacattcaagaacttcaacaaaaggttgagaagatgctttccgaggtcacagacgaaaagcagaaagaaaaagtccacgagtatggacccgcatgcaaaaagatcttcggtgcgacaacactgcaacatcatcgacgaaggaggcatcatttcacccttgaaagtagtctagatacccatctgaaatggctcagtcaggaacagaaagatgaattgttaaaaatgaagaaagatggaaaaacaaagaaagagctcgaggcgaaaattcttcattactatgacgaactcgaaggagatgctaaaaaagaggcaactgagcatttgaaaggcggatgcggcgaaattcttaagcatgttgttggggaagagaaggcagcagagctgaagaacctcaaagactcgggagcaagcaaagaagaactgaaagccaaagtcgaagaggcgcttcatgcagtgaccgacgaggagaagaagcaatacattgccgattttggaccagcatgcaagaaaatctatggtgtacatacttcgcgacgaaggaggcatcatttcacccttgaaagcagtctagatacccatctgaaatggctcagtcaggaacagaaagatgaattgttaaaaatgaagaaagatggaaaggcaaagaaagagctcgaggcgaaaattcttcattactatgacgaactcgaaggagatgctaaaaaagaggcaactgagcatttgaaaggcggatgcgccgaaattcttaagcatgttgttggggaagagaaggcagcagagttgaagaacctcaaagactcgggagcaagcaaagaagaactcaaagccaaagtcgaagaggcgcttcatgcagtgaccgacgaggagaagaagcaatacattgccgattttggaccagcatgcaagaaaatctatggtgtacatacttcgcggcgaaggaggcatcatttcacccttgaaagtagtctagatacccatctgaaatggctcagtcaggaacagaaagatgaattgttaaaaatgaagaaagatggaaagacaaagaaagatcttcaagctaaaattcttcattactatgacgaactcgaaggagatgctaaaaaggaggcaactgagcatttgaaggacggatgccgcgaaattcttaagcacgttgttggggaagagaaggaagcagagctgaagaaactcaaagactcgggagcaagcaaagaggaagtgaaagccaaagtcgaagaggcacttcatgcagtaaccgacgaggagaagaagcaatatatcgccgatttcggaccagcatgcaagaaaatctttggtgcagcacatacttcgcgacgaaggaggcatcatttcacccttgaaagtagtctagatacacatctgaaatggctcagtcaggaacagaaagatgaattgttaaaaatgaagaaagatggaaaggcaaagaaagaactcgaggcgaaaattcttcattactatgatgaactcgaaggagatgctaaaaaagaggcaactgagcacttgaaaggcggatgccgcgaaattcttaagcatgttgttggggaagagaaggcagcagagctgaagaacctcaaagactcgggagcaagcaaagaagaactcaaagccaaagtcgaagaggcgcttcatgcagtgaccgacgaggagaagaagcaatacatcgccgattttggaccagcatgcaagaaaatctatggtgtacatacttcgcgacgaaggaggcatcatttcacccttgaaagtagtctagatacacatctgaaatggctcagtcaggaacagaaagatgaattgttaaaaatgaagaaagatggaaaggcaaagaaagaactcgaagcgaaaattcttcattactatgatgaactcgaaggagatgctaaaaaagaggcaactgagcacttgaaaggcggatgccgcgaaattcttaagcatgttgttggggaagagaaggcagcagagctgaagaacctcaaagactcgggagcaagcaaagaagaactcaaagccaaagtcgaagaggcgcttcatgcagtgaccgacgaggagaagaagcaatacatcgccgattttggaccagcatgcaagaaaatctatggtgtacatacttcgcgacgaaggaggcatcatttcacccttgaaagtagtctagatacccatctgaaatggctcagtcaagaacagaaagatgaattgttaaaaatgaagaaagatggaaaggcaaagaaagaactcgaagcgaaaattcttcattactatgatgaactcgaaggagatgctaaaaaagaggcaactgagcacttgaaaggcggatgccgagaaattcttaagcatgttgttggggaagagaaggcagcagagctgaagaacctcaaagactcgggagcaagcaaagaagaactcaaagccaaagtcgaagaggcgcttcatgcagtgaccgacgaggagaagaagcaatatatcgccgatttcggaccagcatgcaagaaaatctatggtgtacatacttcgcgacgaaggaggcatcatttcacccttgaaagtagtctagatacccatctgaaatggctcagtcaagaacagaaagatgaattgttaaaaatgaagaaagatggaaaggcaaagaaagaactcgaagcgaaaattcttcattactatgatgaactcgaaggagatgctaaaaaagaggcaactgagcacttgaaaggcggatgccgagaaattcttaagcatgttgttggggaagagaaggcagcagagctgaagaacctcaaagactcgggagcaagcaaagaagaactcaaagccaaagtcgaagaggcgcttcatgcagtgaccgacgaggagaagaagcaatacatcgccgattttggaccagcatgcaagaaaatctatggtgtacatacttcgcgacgaaggaggcatcatttcacccttgaaagtagtctagatacccatctgaaatggctcagtcaagaacagaaagatgaattgttaaaaatgaagaaagat

SEQ ID NO: 32 - DNA, 393 bp, Acanthocheilonema viteae
gaacattcaccgccacgctattttcggccgcatctaagctggcttactgatgcccaaaaggatgaggtgctgaagatggaggttgaaaacaaagcaagagcagatattcaagggaagatattacatttctatgaagatttaaacgaggaagcaaaaaaagaagcagctgaattccttaacggtgcatgttacgatatcactgtgcacgtttttggtgatgaaaaagcagaagaactgaagaaagtgagagaatcgactggtgttagtgatgagattcgacgcaaaatggatggaatgattgatgaaattgaggatgaagaccaaaagacaaaagcacaagaatatggcccaatttgccaaaatatttttcttcattatcaacgcaaacatcgt

SEQ ID NO: 33 - DNA, 4902 bp, Dictyocaulus viviparus
ggtttaattacccaagtttgagaacattcgtattgttccgaccgaagagatcctccgtcgaagctgataaggctacgagcgataatttagttgcttctgtggcagccatgaagtcgaccagctttattacattgctactgctttcatatttcatagtagaggctcattctagtatatttcactgggacgatgaaagattgtttaaacatgatgacactcatagctggttaacggatgtacaaaaagctgaacttgaaacgcttaagcaccaaccaatacaattacgagacaaaacactagaattctacaatcaactgcccacaaatgaaaaggcaatatgggataaattttacaccaaatattgcgtagtatggcttaaagaggtggcgtctgatgaggaaatcggcaaacttaaagagttagaatcagaaaagaacaaagaagcactattgactagtatctattcattcaaagatcggcttgatgaagtagatcaaaggaaggtcgaattatggaaggaaacttgtgatgaatatgtaacgaaaggcttgtcacgaaaacgtcgtgacagtaataagaattttgaagagttcatttattggatgaccgacgaacaaaaacaatcaatgaacgatatgaaaacggccggcaaatcattcaatgagattcataaggaaggaagaaagtatttcaaagctttaacgattgataaacaatcatcgcttaaggaacaatttaaggacaagtgtaagaaatacttcatgcaaattgccaattcggatgaggtagagaagattaaatcattgaatgatgatgaaatacgccatgtagtaaaaaatgcagtagcgcgtctaaatggtgaggataaggagttcgccgttaagatggagacgctctgtgaagatgtattggcgtttaaggctcgtaaaaatgatattgacgataaaatcaatagaagattatcgtggatgaccgacgagcagaagcaggtagttaaacaactatacgctgatggaagatctcaagcagatatccgtgcgaaaatcttcgaattcttaagttctatcgacggaccagctggtgttgctgctaaagcccaaattcaaaaagagtgttacaaatggatggaagaggttgcaactgctgaagaaattgcagctttacatgaattgcatgaaatcgatcatgatggatgcagaagaaaagtacgagaattcatcggacgacttccagaagacagaaagctagaagtggagaaagatcttccgttttgcgagaagatatggtaccgtgatcatggtgatcataattcccataagcatggtgcacatcatcatcatcgtcatctagctgtcagacgtagacgacatctatatgccatagaaaagtttctggattggttaaaacccgaacaaaaacatgagctggaaaaaatagaaaatagtggagcacatttcgatgatgttatcgctgaagtgaaaaaattctatggtttattgcctgaagaaaagaaaatcgaattgaaagccaaatttaagtcacaatgctatgattgggtgaaagaggtagctacatccgaagaaatgaatgatattatgaaaatgcatgagtcaaaaaatcattccgatttgatgaaaagactaaccgaactcgaaaatcgtctaactgaagatcagaaacacacaattgaacacgtacgagaagtgtgtttaggtctttgggaggtacagaacaccaacaaacaacataagcagagtcttgaggaggctatggatgcttatctttcgtggatgacggacgaagataaagaaaaagtaaaagcaatttatgaaaccagtaatcgacaaacattttacgatgaaattctaaaaattatggaatcatccgaagatgaagttaaagcaaaagcaacagaaaaattagaagcagcatgtaaacattatggaacgaatattttgggtgaggaaaatgttgatattataagggagatgaaaaaaaatggagctacttttgaggaaatatcaaacagagttgatgagttaattgaaggtattacggattctgatcgtaaagaaaaagcttaccgtatgtcgaaactctgcaaaaaaatatacagtttaggacattcaaaacaacttcaacaatatgattttgaaaatgtactgcaaaagtaccttacttggttagacgattcgcaaaagaatgaattgaggacaatgagtgataataaagaaaaaatttacaaaaaaataattgattactttgatggaacaattggtgaagtgaaagaaaaagcagttgaagaattgcaattagcatgcaaccactatatcaaaagcattgttggtgaagaaaaagctatggaaataaagcagttgaaagaagaaggcaaatcttcggaagaaatagctaaaaaggttgaagacgttatcaatcaaatttctgatgaaagcatcaggagtagagcagatgaagctcttcttgtttgcaagagaattttcggtatcgtaaaacgattgcgaagagacaatagtgaaattcattcattagaagaggcaatggaaagatatctaacttggttgtcggatgaccagaaaattgtaatcaaatccatttatgatgtaaatgatcgtaaagtactttatgagaaaattatggaattctttgatgatgctataggagaaacgaaacaaaaagcagctaaagagcttaaggatgcttgcaagcattacgtgaaagatttaataggtgaagaaaatggaaatttattaagagaaatgaaggaaaacggtgcctcaaatgaagctattgcaacaaaagttgaggaaatgattgaagccattactgatgaaaccaagagagcacaagctatgagagcatctacgtcatgcagaaaggtatacggagttgttcagcgcttcaggagagatcatcaccatgaacataatctagacgaggcattggagaagcacttcacttggttaaacgaagaacagaaatctcagctgaagacaatctatgaaagcgaagaccgcgaagctctacataagaaagtgtgggaatttttcgaagcaggagccggacttcgagcatcaaatgcttccaagaagatttatggtgtcgctaagcgtttcagaagagatcatcaccatgaacataatctagacgaggcattggagaagtacttaacttggttaaacgaagaacagaaatctcagatgaagacaatctatgaaagcggagaccgcgaagctctatacaagaaagtgttggaatttttcgaagcagcaaccggagaagtgaaagaaaaagctgctgttgaacttaaatcagcttgcagacattacattaaagactacattggagacgagaaagcggaaaagataaaagagatgaaagaaagtggagtgagcaccgaagagatttccaaaaaagtggacgaattcattgcgatgatcaccgacgatgaaaagaaagcaaaagcacttcgagcatcaagtgcttgcaagaagatttatggtgtcgctaagcgtttcagaagagatcatcatcatgaacataatctagaagaggcattggagaagtacttaacttggttaaacgaagaacagaaatctcagatgaagacaatctatgaaagcggagaccgcgaagctctatacaagaaagtgttggaatttttcgaagcagcaaccggagaagtgaaagaaaaagctgctgttgaacttaaatcagcttgcagacattacattaaagactacattggagacgagaaagcggaaaagataaaagagatgaaagaaagtggagtgagcaccgaagagatttccaaaaaagtggacgaattcattgcgatgatcaccgacgatgaaaagaaagcaaaagcacttcgagcatcaaatgcttgcaagaagatttatggcgtcgctaagcgtctcagaagagatcatcatcatgaacacaacttggaagaagcaatgggaaaatatttgtcttggatgagcgatgaacaacaggctcaagtgaaaaaaatctatggaactggtgatcgactagcgacttataataaagtgatggaattatttgaatctgtgccaagtgacgagaaagagaaagccactagtcaactaaaagctgcttgtagacattacatcaaagatttcattggtaaagataatctcgcagtcattaaagaaatgaaagaaagcggtgcgacaaatgaagctatcggagaaaaaatagatgaattcattgctggcttagatgatgaacaaaaaaaagcacaagctcaacgagcagcatcagcatgtaagaagatctatggagtgaaaagtcgaaaacgacgagaacattacgaaatagacgtggatgaagcaatatcgaaatatttaacatggttaaatgaggaacaaaaagccgaaattaagcaactgaaagagaaagacgaaaaacaaacaatcggtaagaaaattatggagttttttgagttgactagtggtgacgataaagaaaaagctagagaacaacttaaagccgcttgtaagcattatgttaaaatgtatgtcggtgaagaaaaagccgcagaacttaaaaaattgaaagattctggcatttcactagaagaaatgtcgaaaaaagtcactgaaacgattgaaacgatagaagatgaagcagtacgagcgaaagcacgacgaattcattcgtattgtcagagaatcttcggcatcacgaaagcacgacgtcatctcgctatgaaacatcatcgattttatgacgattgacgttgaatttcatctatgatttcattgatagccatgtgatatcatatcagttattttttgtatctgctttttaatgtgaaatatatcttataattagtaatgaaatgaattgaaatcgctt

SEQ ID NO: 34 - DNA, 1421 bp, Ostertagia ostertagia
cattcacttgaagacgcaatggggaagtatttgacgtggttaactgatgaccaaaaggaggaggtcaagtcactttacacggatgaaggcagaggtgccgtgtatgataaaattatggagtacttcgatgaggctacaggtgacaggaaggagaaagcagcaaaggaattgaagggcgcttgtaagcattatgtgaaagacctgattggcgagaaaaatggtgagatgatcaaagagatgaaagaaaacggggcttcaaatgatgccatcgcaacaaaagtcgaagagctgattgaagcaatcgcagacgataagaaaaaggcgcaggcacttcgagcatctgcaaactgtaggaaaatttatggagtggctcgtcgattcagaagagatcaccatgaacacaacttggaagaagccatggagaaatatctcacctggctgaatgatgaccaaaaagaggaggtcaagaagttgtacggcgcaggcgacaaacaagccatgtacaagaaggtgatggaaatctatgacagtgtttctggtgacgtgaaagagaaggccaccgtcgagctgaaggcagcctgcaggcactatgttaaggactctatcggtgaggagaatgccgagaagctcaaggagatgaaggaaagtggagctaccccggaagccattgctgccaaagttgaggaattcatagctgccatcaccgatgagaagaagaaagcacaagcagaacgagccgcagtagcgtgtaaaaagatatatggtgtggcccggcgcctaaagagggagcatcacgaacataaccttgaggaagccatggaaaagtacctcacttggttgaatgacgaacaaaaggaggaggtgaagaaaatctatggaaccggagatcgtatcgcagtcgagacgaaagtactgcagatgtttgagaacgcctcaggtgacgtcaaggagaaagcatctgtacaactgagagccgcttgcaaacactacatcaaagagtacattggagacgagaacgtggctaaaatcaaggagatgaaagacagtggagcgagcaatgaggcgatgtctgctaaaattgacgagttcatcgctgccatacctgagaaggagagaaaagagaaagctgaacgggtagcagcgtcatgcaagaaggtttatggggtgaaaagccgaatgagaaggtatccggcgcgcagcacgcgttccacatagagggttacaatagacgtgcattgagagcccagcgtcatctggctgaaaggcgtcgacgaaatgaggctaaggttcatttcttagatatctgaataatagcataccaatttatgtatctaatgttgatcgtgaattttgtaacagacaccctactaggtcaagatgatcttttttcttaatattgtccacaataaatatttgacggg

(19) United States
(12) Patent Application Publication
(10) Pub. No.: US 2007/0053920 A1
(43) Pub. Date: Mar. 8, 2007

(54) Nematode polypeptide adjuvant

(76) Inventors: Andrew Heath, Sheffield (GB); Peter Laing, Sheffield (GB)

Correspondence Address: KLARQUIST SPARKMAN, LLP, 121 SW SALMON STREET, SUITE 1600, PORTLAND, OR, 97204, US

(21) Appl. No.: 10/543,731

(22) PCT Filed: Jan. 21, 2004
(86) PCT No.: PCT/GB04/00149

§ 371 Date: Mar. 23, 2006

(30) Foreign Application Priority Data
Jan. 22, 2003 (GB) 0301433.9

(51) Int. Cl. A61K 39/02; A61K 39/00

(52) U.S. Cl. 424/185.1; 424/234.1

(57) ABSTRACT

The invention relates to a set of novel immunological adjuvants based upon so called “polyladder” proteins of nematode worms. These proteins are typified by repeating units separated by a protease cleavage motif of RX(K/R)R or RXFR where R is ariginine, X is any amino acid, K is lysine and F is phenylalanine. These motifs are preceded by a cysteine residue at around 7, 8 or 9 residues upstream. Polyladder proteins or fragments of polyladder proteins may be used as immunological adjuvants either mixed with, or conjugated to a vaccine antigen, and will strongly enhance the immune response against the antigen. Conjugation may take the form of a genetic fusion between adjuvant and antigen. Antigens may be derived from pathogens, or may be tumour antigens, autoantigens, or antigens of other kinds. Vaccines may be used for prophylaxis or therapy.

The invention relates to a polypeptide adjuvant for use in vaccine compositions.

An adjuvant is a substance or procedure which augments specific immune responses to antigens by modulating the activity of immune cells. Examples of adjuvants include, by example only, Freunds adjuvant, muramyl dipeptides, liposomes. Adjuvants may also be antibodies to receptors expressed by immune cells which act either agonistically or antagonistically. An adjuvant is distinct from a carrier which is often used to enhance an immune response to an antigen.

A carrier is an immunogenic molecule which, when bound to a second molecule augments immune responses to the latter. Some antigens are not intrinsically immunogenic (i.e. not immunogenic in their own right) yet may be capable of generating antibody responses when associated with a foreign protein molecule such as keyhole-impet haemocyanin or tetanus toxoid. Such antigens contain B-cell epitopes but no T cell epitopes. The protein moiety of such a conjugate (the “carrier” protein) provides T-cell epitopes which stimulate helper T-cells that in turn stimulate antigen-specific B-cells to differentiate into plasma cells and produce antibody against the antigen. Adjuvants that are protein ligands of immune cell receptors may have the quality of both adjuvant and carrier, the latter depending on their content of ‘foreign’ polypeptide sequences that can be recognised by T-cells of the immune system. The new adjuvants described in the present invention, may have both properties.

Polyprotein antigens or polyladder proteins are produced by a number of parasitic and free-living nematode species. These polyproteins are generally composed of multiple units arranged in direct tandem arrays, and the proteins are generally synthesised as large precursor proteins which are cleaved by proteases to yield smaller fragments as a “ladder” with steps of around 15 kDa, reflecting increments in the denominator molecular mass of the individual domains. The last 4 amino acids of each such unit are usually comprise a protease-labile RX(K/R)R (or occasionally RXFR) motif. In addition these motifs are preceded by a cysteine residue 7, 8 or 9 residues upstream (N-terminal of the motif) (McReynolds et al. (1993) Parasitology today 9 403-406), which may serve to distance the protease cleavage site from the body of the protein domain.

Some parasite polyproteins (such as the DiAg proteins of Dirofilaria immitis) are strong immune stimulators, giving rise to production of antigen-non-specific IgE, and play important roles in the evasion of the immune response by parasites, by interfering with the production of parasite-specific IgE.

One of the most important developments in medical science in recent history is the production of vaccines which provide prophylactic protection against a wide variety of infectious diseases. Many vaccines are currently in development for prevention and treatment of yet other categories of disease, including autoimmune, neurodegenerative diseases and cancer. The present invention also applies to these additional categories of disease. Vaccines for the prevention of infectious diseases are, in many instances, made from inactivated or attenuated forms of the disease causing agent (or pathogen) which are injected or otherwise administered into a the recipient in order to prevent infection with the natural form of the pathogen. The recipient individual may respond by producing a humoral (antibody) response, a cellular (e.g. a cytolytic T cell, CTL) response, or both.

The development of so-called subunit vaccines (in which the immunogen is a defined molecular fragment or subunit of an infectious agent, or a tumour antigen) has been the focus of considerable medical research. The need to identify candidate molecules (e.g. proteins or polysaccharides) useful in the development of subunit vaccines was originally based on the need for increased safety, and is also driven, in the case of vaccines against bacterial infections, by the increasing problem of antibiotic resistance. However, subunit vaccines tend to be less immunogenic than are vaccines based on whole organisms, and are more highly dependent on ‘adjuvants’ in order to elicit an efficacious immune response that protects against infection with the target organism (or which generates an effective anti-tumour immune response).

We describe a family of proteins which act as immunological adjuvants to enhance immune responses against various prophylactic and therapeutic vaccines. The adjuvant system has potent action in stimulating immune responses against vaccine antigens. The new adjuvant system is particularly applicable to subunit vaccines, but is also readily applicable to other vaccine types (including vaccines based on whole organisms, nucleic acids etc.).

Polyladder proteins of nematodes are known to be highly effective at inducing IgE responses. (Tomlinson et al. J. Immunol. 143 2349-2356 (1989); Paxton et al. Infect Immunol. 61 2827-2833 (1993). However, some polyladder proteins (such as DiAg of Dirofilaria immitis) appear to subvert the appropriate immune response by generating antigen-non-specific IgE, which is incapable of binding to DiAg itself or to the parasite (Tezuka, H et al. Infection and Immunity, July 2003, 3802-3811), and may interfere with parasite elimination by arming mast cells and eosinophils with irrelevant IgE, to the exclusion of parasite-specific IgE. Moreover, IgE responses are generally regarded as an undesirable outcome of vaccination (at least in the case of vaccines against agents other than parasites), because IgE antibodies are associated with allergic reactions that can be dangerous and even life-threatening (e.g. anaphylaxis can occur in a subject who encounters an antigen, if they have pre-existing IgE antibodies specific for the antigen). Moreover, there is a finite risk that polyladder proteins could boost ongoing allergen-specific IgE responses in human subjects, or interfere with desirable immune responses to parasites if used in vaccine materials. In summary, the elicitation IgE responses by polyladder proteins and the elicitation of non-specific IgE responses that may interfere with parasite elimination or exacerbate allergic disease are all contraindications for the use of parasite polyladder proteins as vaccine constituents or adjuvants.

Surprisingly, we now disclose that physical association, e.g. by conjugation or particulate co-formulation, of the polyladder proteins, (or preferably single repeat units of these proteins), is a means to achieve a very strongly enhanced immune response against the associated antigen, in the absence of a strong non-specific IgE response. We disclose that the physical association of polyladder proteins (or preferably individual single domain moieties of the polyladder proteins) with an antigen against which an immune response is desired can convert the potentially dangerous non-antigen-specific IgE response to the polyladder proteins into a beneficial adjuvant effect, giving rise to desirable antigen-specific antibodies against the antigen. We also disclose how the resulting antigen-specific immune response (to the polyladder-domain-associated vaccine antigen) can be biased towards IgG production (suitable for example for the elimination of bacterial pathogens), and towards the production of Th1 type T-cell responses suitable for the elimination of intracellular parasites such as viruses (and some parasites), and biased away from potentially dangerous IgE responses. We also disclose how polyladder proteins or protein domains can be used to generate cytolytic T lymphocyte (CTL) responses against the associated vaccine antigen, even when the antigen is administered in a non-particulate form. Furthermore, we disclose how polyladder protein domains (even domains of DiAg polyladder protein) can be used to generate desirable antigen-specific IgE responses against antigens (e.g. parasite antigens other than DiAg and polyladder proteins) that are physically associated with them. DiAg and related proteins have been shown to bias the immune system away from pathogenic Th1 responses responsible for autoimmune type-I (insulin dependent) diabetes in mice and are advocated as therapeutically useful for the treatment of Th1 based autoimmune diseases (Imai, S. et al. Biochem. Biophys. Res. Comm. 286:1051-1058). Surprisingly therefore, in a further aspect of the present invention, we now disclose how DiAg and related proteins can be used to treat allergic diseases, which are the polar opposite (Th2) of the T-cell profile (Th1) involved in the pathogenesis of allergic diseases. The compositions and methods necessary to create these novel utilities of DiAg proteins are described below.

According to an aspect of the invention there is provided a polypeptide wherein said polypeptide comprises:
i) an amino acid motif consisting of the amino acid residues
   RXK/RR wherein R is arginine, X is any amino acid residue, and K is lysine; and/or
ii) an amino acid motif consisting of the amino acid residues
   RXFR wherein F is phenylalanine and further wherein said motif(s) is preceded by a cysteine amino acid residue about 7-9 residues amino terminal to said motif(s) which polypeptide can be modified by addition, deletion, or substitution of at least one amino acid residue
      for use as an immunolgical adjuvant.

According to an aspect of the invention there is provided an adjuvant comprising a polypeptide encoded by a nucleic acid molecule wherein there is at least one motif of the sequence RX(K/R) R (wherein R is arginine, X is any amino acid, K/R is lysine or arginine, and R is arginine); or RXFR motif (where F is phenylalanine) preceded by a cysteine residue 7, 8, or 9 residues N-terminal of this RX(K/R)R or RRFR motif.

Preferably said polypeptide is encoded by a nematode nucleic acid molecule.

In another embodiment of the invention there is provided an adjuvant which is a fragment of said protein, and preferably a lymphocyte binding fragment.

In another embodiment of the invention there is provided an adjuvant with at least 70% homology to said protein, or to a fragment of said protein, and preferably a lymphocyte binding fragment.

According to a further aspect of the invention there is provided a vaccine composition comprising at least one polypeptide wherein said polypeptide comprises;
i) an amino acid motif consisting of the amino acid residues RXK/RR wherein R is arginine, X is any amino acid residue, and K is lysine; and/or
ii) an amino motif consisting of the amino acid residues RXFR wherein F is phenylalanine and further wherein said motif(s) is preceded by a cysteine amino acid residue about 7-9 residues amino terminal to said motif(s) which polypeptide can be modified by addition, deletion, or substitution of at least one amino acid residue and at least one antigen to which an immune response is desired.

In a preferred embodiment of the invention said polypeptide is mixed with said antigen.

In a further preferred embodiment of the invention, said polypeptide is conjugated, associated or crosslinked to said antigen.

In a further preferred embodiment of the invention said polypeptide comprises a Dirofilaria immitis protein, Neutrophil chemotactic factor (NCF), or lymphocyte binding fragment thereof, or homologue thereof, or lymphocyte binding fragment of homologue thereof.

In a preferred embodiment of the invention said polypeptide comprises an amino acid sequence selected from the group consisting of: SEQ NO. 1; 2; 3; 4; 5; 6; 7; 8; 9; 10; 11; 12; 13; 14; 15; 16; 17; 18; 19; or a polypeptide which is at least 50% homologous, and more preferably 70% homologous, and more preferably still, 90% homologous to a sequence from this group.

In a preferred embodiment of the invention the length of said polypeptide is of at least 20 consecutive amino acids identical in sequence to at least a 20 amino acid portion of a sequence selected from SEQ D No: 1; 2; 3; 4; 5; 6; 7; 8; 9; 10; 11; 12; 13; 14; 15; 16; 17; 18; or 19;.

In a preferred embodiment of the invention said polypeptide is a lymphocyte binding fragment of such a protein.

In another embodiment of the invention said polypeptide is encoded by a nucleic acid molecule comprising a nucleic acid sequence selected from a group consisting of, SEQ ID No: 20; 21; 22; 23; 24; 25; 26; 27; 28; 29; 30; 31; 32; 33; or 34; or a nucleic acid molecule which hybridises under stringent hybridisation conditions to said nucleic acid molecule and which encodes a polypeptide with immunolgical adjuvant activity.

Preferably said nucleic acid sequence has at least 50% homology to a sequence from this group, or preferably at least 70% homology to a nucleic acid sequence from this group, or more preferably at least 85% homology to a sequence from this group.

In a further embodiment of the invention said polypeptide is encoded by a 60 nucleotide portion of a nucleic acid sequence selected from a group consisting of: SEQ ID No: 20; 21; 22; 23; 24; 25; 26; 27; 28; 29; 30; 31; 32; 33; or 34.

In a further preferred embodiment, adjuvant protein consists of a protein homologous to parts of a nematode ladder protein between, but not including the whole RX(K/R)R or RRFR sequence, and most preferably avoiding the R, K (and occasionally F) residues of this sequence.

In further embodiment of the invention, RX(K/R)R protease cleavage motifs (such as those underlined in sequence (1) are mutated or are not included in the adjuvant protein.

A useful example of this mutated sequence is a sequence wherein R and K residues are replaced by glycine ‘G’ residues. A second example is one in which the R and K residues are replaced by serine residues ‘S’. A third example is where the R and K residues are replaced by G or S in any permutation (e.g. GXGS, SXSG, GXSG etc.)

Usefully the linker may be longer than occurs naturally in polyladder proteins, (e.g. up to 30 residues), most preferably 5-20 amino acid residues and typically lacks any strong propensity to secondary structure (such as helical propensity or tendency to form beta-sheet), and typically lacks residues capable of cleavage by trypsin-like enzymes (principally K and R).

This lack of propensity to form secondary structure may usefully be engineered by incorporation of proline residues ‘P’ at intervals, e.g. every third residue or every tenth residue, but more preferably every 4^{th}, 5^{th} or 6^{th} residue. Alternatively the prolines may be randomly distributed in the fusion zone of the sequence of the fusion protein such that a small linker sequence of 5 residues would contain one proline, whereas a larger sequence of 15 residues would contain 3 or four prolines.

The purpose of the linker sequence is primarily to join the two protein moieties in the fusion protein (namely the polyladder protein moiety and the antigen moiety), in a manner that is relatively stable to proteases (unlike the situation in the native polyladder protein sequence, where the boundaries between domains are highly protease labile) however a second function of the sequence is to allow the protein moieties to fold during biosynthesis into their native domains upon which their functional attributes (adjuvanticity and antigenicity/immunogenicity) depend. While the polypeptide linker used can take many forms, it is important that the linker does not contain sequences from human autoantigens (or autoantigens from the animal to be immunized). Thus putative linker sequences should be typically screened against databases in order to ensure that the linker has no significant homology to human proteins, especially proteins known or suspected to play a role in the aetiology of autoimmune diseases such as glutamate decarboxylase, insulin, thyroglobulin, thyroid peroxidase, islet cell autoantigens, parietal cell autoantigens, kidney autoantigens, myelin basic protein, myelin associated glycoprotein, myelin oligodendrocyte glycoprotein.

An exception to this general rule would be the case where such proteins are non-organ specific in their distribution in the body, and abundantly expressed—such as the blood proteins albumin and the immunoglobulins. For example, the hinge region of IgG would make a good linker sequence for the said fusion protein, since it is not especially protease labile. Likewise the hinge region of IgA would make a good linker sequence since only very few proteases (e.g. the meningococcal IgA protease) are able to cleave this sequence, despite its exposure (in the three dimensional structure of IgA) and flexible nature of the sequence. RX(K/R)R of RXFR motifs may be not included in the adjuvant protein, because the protein used as an adjuvant commences downstream (carboxy-terminal) of one cleavage motif, and ends upstream (amino terminal) of the next one. The start and end of the adjuvant protein can also be internal to the protease motifs.

In a preferred embodiment of the invention said polypeptide is conjugated or crosslinked to said antigen with protein cross-linking agents such as glutaraldehyde or EDC (ethylcarbodiimide a water soluble carbodiimide), or preferably with heterobifunctional reagents such as MBS and others described in the literature, and in the catalogue of the Pierce Chemical Company of Rockford, Ill., USA, or the catalogue of Molecular Probes Inc. of Eugene, Oreg., USA.

In a preferred embodiment of the invention said adjuvant is produced as a fusion protein with said antigen, by in frame fusion of nucleic acids encoding antigen and adjuvant using methods of in vitro DNA recombination and cloning that are well known in the art.

In a further preferred embodiment of the invention said polypeptide and said antigen are encapsulated in synthetic microparticles or nanoparticles (e.g. polylactide-glycolide or ‘PLG’), liposomes, or immune stimulating complexes (ISCOMs).

Particulate formulation is desirable because it directs antigens to antigen-presenting cells favouring a Th1 profile of immune response against the antigen, and countering any tendency of the polyladder protein moiety towards expression of Th2 profile and IgE production. Such modes of formulation will be useful for the stimulation of desirable cell-mediated and IgG antibody responses against the antigen. Particulate formulation also allows the facile incorporation of additional materials designed to bias the immune response in the direction of Th1. Such materials would typically include antibodies against IL10 and IL4, Th1 cytokines such as IFN-gamma, and CpG DNA.

Most favourably, particulate formulations will comprise both polyladder protein moiety and antigen against which an immune response is desired formulated in the same particle such that each particle in a formulation carries both entities as payloads. Such formulation ensures that both materials be taken up by any given single antigen presenting cell, and maximises the Th1 biasing effect of particulate formulation for the payload antigen, even when such antigen and polyladder domain (and optional Th1 biasing materials mentioned in the paragraph above) are not otherwise connected, except by being both present in the same particle.

Particulate formulations preferably have a significant degree of surface exposure (5-10%) of polyladder protein moiety and antigen moiety. Generally such levels of exposure are achieved by default in the particulate formulation process. In cases where such exposure is not achieved, the aforesaid protein moieties can be conjugated to the surface of the particle by covalent conjugation. Surface exposure of the antigen moiety favours the stimulation of antigen specific B-cells and is helpful for antibody responses against the antigen.

These particles are typically in the size range 150 nanometres up to 10 micrometres across. More preferably they are in the range 200 nanometres up to 2 micrometres.

In a preferred embodiment of the invention said polypeptide and antigen are co-adsorbed or co-precipitated onto aluminium or calcium salts, such as aluminium hydroxide gel or calcium phosphate.

In a further preferred embodiment of the invention said polypeptide is encoded by a nucleic acid molecule which is part of a vector wherein the expression of said polypeptide is operably controlled by a promoter.

In a preferred embodiment of the invention said antigen is encoded by a nucleic acid molecule. Preferably said nucleic acid molecule is part of a vector wherein expression of said antigen is operably controlled by a promoter.

In a preferred embodiment of the invention said polypeptide and said antigen are encoded by the same nucleic acid molecule. Preferably said nucleic acid molecule encodes an in frame fusion of said polypeptide and said antigen.

In a preferred embodiment of the invention said in frame fusion includes a linker nucleic acid molecule encoding a flexible linker sequence (e.g. encoding oligo serine or glycine, or serine-glycine combinations with the number of residues).

Preferably said vector is a “shuttle vector”, capable of propagation in E.coli and of expression of the fusion protein in mammalian cells via a suitable promoter e.g. CMV or other eukaryotic promoter.

According to a further aspect of the invention, the adjuvant protein domain (e.g. from NCF of D. immitis) is represented in several copies—(most preferably 1, 2 or 3) as co-linear fusions with antigen (in single copy) as part of the same polypeptide chain. Alternatively, the adjuvant protein domain is present as a single copy fused to an antigenic protein which forms oligomers (e.g. dimers, trimers, tetramers etc.). In this latter construct the adjuvant protein domain becomes oligomeric once the antigen protein oligomerises. Most conveniently, a single copy of the adjuvant protein is made as an in-frame fusion with an oligomerising protein from the infectious agent against which a vaccine is designed to protect, such as the influenza hemagglutinin or the HIV coat glycoprotein gp120. Alternatively, the adjuvant protein domain in single copy is fused to an antigen that does not oligomerise. In such cases, oligomeric forms may be created by incorporation of a protein moiety (such as a coiled coil) with a natural tendency to oligomerise. Examples of suitable oligomerising moieties are the paired helix coiled-coil structures of streptococci (e.g. the M-proteins) which form dimeric coiled coils; also trimeric helical protein moieties may also be used. One example is the stem part of type-2 membrane proteins such as CD23. Artificial coiled coil peptides that have been designed in order to study the assembly characterisitics of coiled coil proteins would also be suitable. Most preferably the degree of oligomerisation is the trimer, since this reflects the postulated natural state of the D. immitis protein. In instances where the adjuvant domain is represented in multiple copies, the most preferred embodiment will be the natural repeat structure of the D. immitis protein. In cases where a domain is repeated as part of a single polypeptide chain, it is most preferable to exploit alternative codon usage at the DNA level, in order to avoid direct repeats in the DNA sequence—which otherwise would give rise to problems of homologous recombination and deletion during propagation of the encoding DNA.

A conjugate may be formed by the use of cross-linking agents to link adjuvant to antigen.

Alternatively, conjugates may be translational fusions between adjuvant, and antigen.

In a further preferred embodiment of the invention said composition comprises a carrier.

In a still further preferred embodiment of the invention said composition comprises a second adjuvant.

In a preferred embodiment of the invention, said antigen is a T-cell dependent antigen.

In an alternative preferred embodiment of the invention said antigen is a T-cell independent antigen such as bacterial capsular polysaccharide (e.g. of Streptococcus pneumoniae, Neisseria meningitidis, Haemophilus influenzae or Group B Streptococcus).

In a preferred embodiment of the invention said antigen is derived from a pathogenic bacterium.

Preferably said antigen is derived from a bacterial species selected from the group consisting of: Staphylococcus aureus; Staphylococcus epidermidis; Enterococcus faecalis; Mycobacterium tuberculsis; Streptococcus group B; Streptoccocus pneumoniae; Helicobacter pylori; Neisseria gonorrhoea; Streptococcus group A; Borrelia burgdorferi; Coccidiodes immitis; Histoplasma sapsulatum; Neisseria meningitidis type B; Shigella flexneri; Escherichia coli; Haemophilus influenzae, Chalmydia trachomatis, Chlamydia pneumoniae, Chlamydia psittaci, Francisella tularensis, Bacillus anthracis, Clostridium botulinum, Yersinia pestis, Burkholderia mallei or B pseudomallei

In an alternative preferred embodiment of the invention said antigen is derived from a viral pathogen.

Preferably said antigen is derived from a viral pathogen selected from the group consisting of:: Human Immunodeficiency Virus (HIV1 & 2); Human T Cell Leukaemia Virus (HTLV 1 & 2); Ebola virus or other haemorrhagic fever virus; human papilloma virus (HPV); papovavirus; rhinovirus; poliovirus; herpesvirus; adenovirus; Epstein Barr virus; influenza virus A, B or C, Hepatitis B and C viruses, Variola virus, rotavirus or SARS coronavirus.

In a further preferred embodiment of the invention said antigen is derived from a parasitic pathogen.

In a yet further preferred embodiment of the invention said antigen is derived from a parasitic pathogen selected from the group consisting of Trypanosoma cruzi, Trypansosoma brucei, Schistosoma spp; Plasmodium spp. Loa Loa, Leishmania spp; Ascaris lumbricoides, Dirofilaria immitis, Toxoplasma gondii.

In a further preferred embodiment of the invention said antigen is derived from a fungal pathogen.

In a preferred embodiment of the invention said antigen is derived from a fungal pathogen which is of the genus Candida spp, preferably the species Candida albicans.

In a further preferred embodiment of the invention, said antigen is a tumour specific antigen (e.g. carcinoembryonic antigen, the human polymorphic epithelial mucin, MUC-1, or a hormone or analog thereof involved in hormone dependent cancer, such as gastrin).

In a further embodiment of the invention, said antigen is a ganglioside antigen.

In a further preferred embodiment of the invention said antigen is a human host antigen, such as a hormone, hormone receptor, T cell receptor or sperm antigen.

In a further preferred embodiment of the invention said antigen is a prion protein.

In a further preferred embodiment of the invention said antigen is an amyloid protein or a fragment of an amyloid protein such as the 40 residue amyloidogenic peptide fragment (Aβ) of the amyloid precursor protein of Alzheimer's disease.

In a further preferred embodiment of the invention, said antigen is a toxin such as ricin, or a fragment of a toxin or a toxoid.

According to a further aspect of the invention there is provided a nucleic acid molecule which encodes conjugate wherein said conjugate comprises an antigenic polypeptide translationally fused to a nematode derived ladder protein in which an RX(K/R)R or RRFR motif is preceded 7, 8 or 9 residues upstream by a cysteine residue.

According to a further aspect of the invention there is provided a means to treat allergic disease by administration of a solution or particulate (e.g. liposomal) formulation of a polyladder protein, or part thereof according to the invention (e.g. DiAg).

An advantage of this mode of treatment of allergic disease is that it can be applied to all or any allergic disease, irrespective of the allergen, and even where the allergen(s) may be unknown (e.g. allergic asthmatic conditions). DiAg and related polyladder proteins, can be used to generate unusually large quantities of non-antigen-specific IgE that compete with sites (high affinity IgE receptors) on mast cells and eosinophils, deprive such cells of allergen-specific IgE, and prevent them from becoming activated and releasing inflammatory mediators upon contact with allergen.

For example, because DiAg in human parasite infestations does not give rise to DiAg specific IgE, the risk of anaphylaxis developing as a result of DiAg therapy is minimal. In this aspect of the invention, individual polyladder (e.g. DiAg) domains can be used. Such domains can be administered as protein solutions in pharmaceutically acceptable saline vehicles, or encoded as DNA or RNA in plasmid or viral vectors for mammalian expression., or in liposomal vectors as plasmid constructs being expressible in the body of the vaccinee.

According to a further aspect of the invention, there is provided a method to enhance the immune response against multivalent vaccines, especially multivalent polysaccharide vaccines by co-formulation of a carrier protein-polyladder conjugate or chimeric protein, with conjugates of various antigens, such as polysaccharide antigens with the same carrier protein.

Typically, in this case the two carrier proteins may be the same protein, or may be different, but containing at least one T helper epitope in common with each other. One or both may be a synthetic peptide. An example might be multivalent pneumoccal conjugate vaccine, which consists of a number of different polysaccharides, each conjugated to a mutant diptheria toxoid. By the simple addition into this conjugate mixture of a polyladder protein-diptheria toxoid conjugate, immune responses against all the polysaccharide antigens in the conjugate will be strongly enhanced.

According to a further aspect of the invention there is provided a nucleic acid molecule which encodes conjugate wherein said conjugate comprises an antigenic polypeptide translationally fused to adjuvant of at least 50%, homology, and more preferably at least 70% homology and more preferably still at least 90% homology to sequence from the group comprising: SEQ1, SEQ2, SEQ3, SEQ4, SEQ5, SEQ6, SEQ 7, SEQ8 SEQ9, SEQ10, SEQ11, SEQ 12.SEQ 13, SEQ 14, SEQ15, SEQ 16, SEQ 17, SEQ 18, SEQ 19.

According to a further aspect of the invention there is provided a nucleic acid molecule which encodes conjugate wherein said conjugate comprises an antigenic polypeptide translationally fused to adjuvant where the adjuvant is a protein of at least 20 consecutive amino acids identical in sequence to at least a 20 amino acid portion of a sequence selected from the group comprising: SEQ 1, SEQ 2, SEQ 3, SEQ 4, SEQ 5, SEQ 6, SEQ 7, SEQ 8 SEQ 9, SEQ 10, SEQ 11, SEQ 12. SEQ 13, SEQ 14, SEQ 15, SEQ 16, SEQ 17, SEQ 18, SEQ 19.

In a preferred embodiment of the invention, said nucleic acid molecule is part of an expression vector wherein said nucleic acid molecule is operably linked to a promoter.

In a further preferred embodiment of the invention said vector is selected from the group consisting of: a plasmid; a phagemid; or a virus.

In a further preferred embodiment of the invention said viral based vector is based on viruses selected from the group consisting of: adenovirus; retrovirus; adeno associated virus; herpesvirus; lentivirus; baculovirus.

As used herein, a “vector” may be any of a number of nucleic acids into which a desired sequence may be inserted. Vectors include, but are not limited to, plasmids, phagemids and virus genomes. A cloning vector is one which is able to replicate in a host cell, and which typically is further characterised by one or more endonuclease restriction sites at which the vector may be cut in a determinable fashion and into which a desired DNA sequence may be ligated such that the recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

Vectors may further contain one or more selectable marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase), and genes which visibly affect the phenotype of transformed or transfected cells, hosts, colonies or plaques (e.g., various fluorescent proteins such as green fluorescent protein, GFP). Preferred vectors are those capable of autonomous replication, also referred to as episomal vectors. Alternatively vectors may be adapted to insert into a chromosome, so called integrating vectors. The vector of the invention is typically provided with transcription control sequences (promoter sequences) which mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues which include, by example and not by way of limitation, intermediary metabolites, environmental effectors.

Promoter elements also include so called TATA box, RNA polymerase initiation selection (RIS) sequences and CAAT box sequence elements which function to select a site of transcription initiation. These sequences also bind polypeptides which function, inter alia, to facilitate transcription initiation selection by RNA polymerase.

Adaptations also include the provision of autonomous replication sequences which both facilitate the maintenance of said vector in either the eukaryotic cell or prokaryotic host, so called “shuttle vectors”. Vectors which are maintained autonomously are referred to as episomal vectors. Episomal vectors are desirable since these molecules can incorporate large DNA fragments (30-50 kb DNA). Episomal vectors of this type are described in WO98/07876.

Adaptations which facilitate the expression of vector encoded genes include the provision of transcription termination/polyadenylation sequences. This also includes the provision of internal ribosome entry sites (IRES) which function to maximise expression of vector encoded genes arranged in bi-cistronic or multi-cistromic expression cassettes.

Expression control sequences also include so-called Locus Control Regions (LCRs). These are regulatory elements which confer position-independent, copy number-dependent expression to linked genes when assayed as transgenic constructs in mice. LCRs include regulatory elements that insulate transgenes from the silencing effects of adjacent heterochromatin, Grosveld et al., Cell (1987), 51: 975-985.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc.(1994).

It is known in the art that nucleic sequences are present in vectors known as CpG motifs or ISSs (immune stimulating sequences). These consist minimally of non-methylated CG dinucleotides as a core, although sequences adjacent to the dinucleotide affect the magnitude of the stimulation induced. These ISSs activate antigen presenting cells (APCs) through a toll-like receptor (TLR9). The general aim in DNA vaccination is to include these motifs in the vector, as they enhance the response by activating APCs.

In a further preferred embodiment of the invention said promoter is a tissue specific promoter, such as a muscle specific promoter, allowing intramuscular immunisation with DNA-based vaccines.

Muscle specific promoters are known in the art. For example, WO0009689 discloses a striated muscle expressed gene and its cognate promoter, the SPEG gene. EP1072680 discloses the regulatory region of the myostatin promoter. U.S. Pat. No. 5,795,872 discloses the use of the creatine kinase promoter to achieve high levels of expression of foreign proteins in muscle tissue. The muscle specific gene Myo D shows a pattern of expression substantially restricted to myoblasts.

According to a yet further aspect of the invention there is provided a vaccine comprising a nucleic acid or a vector according to the invention.

According to a further aspect of the invention there is provided a method to immunise an animal to an antigen, comprising administering an effective amount of a conjugate according to the invention sufficient to stimulate an immune response to said antigen.

In a preferred method of the invention said animal is human.

In an alternative preferred method of the invention said animal is selected from the group consisting of: mouse; rat; hamster; goat; sheep, dog or cat.

In a further preferred method of the invention, said animal is immuno-compromised, for example a Hu-SCID-PBL mouse, or a SCID-hu mouse, or a mouse, or other animal otherwise engrafted with human lymphocytes or lymphocyte precursors, such that human antibody and T cell responses can be induced.

Immuno-deficient or immuno-compromised mammals are know in the art. For example, EP0322240 and EP0438053 disclose the grafting of haematopoietic cells into a CID or SCID host organism (see McGuire et al Clinical Immunology and Immunopathology (1975) 3: 555-566) each of which is incorporated by reference. WO9505736, which is incorporated by reference, also teaches the use of SCID organisms and their use as hosts for human cells.

In a still further preferred method of the invention, said animal is transgenic for human immunoglobulin or T cell receptor DNA.

In a further preferred method of the invention said immune response is the production of antibodies to said conjugate.

In an alternative preferred method of the invention said immune response is the production of T-helper cells which recognise the antigen part of said conjugate.

In a further preferred method of the invention, said immune response is the production of cytolytic T lymphocytes which recognise the antigen part of said conjugate.

Preferred routes of administration are oral (e.g. mucosal), intradermal, subcutaneous, intranasal or intramuscular, however the immunisation method is not restricted to a particular mode of administration.

According to a yet further aspect of the invention there is provided an antibody obtainable by the method according to the invention.

In a preferred embodiment of the invention said antibody is a therapeutic antibody.

In a further preferred embodiment of the invention said antibody is a diagnostic antibody. Preferably said diagnostic antibody is provided with a label or tag.

In a preferred embodiment of the invention said antibody is a monoclonal antibody or binding fragment thereof. Preferably said antibody is a humanised or chimeric antibody.

A chimeric antibody is produced by recombinant methods to contain the variable region of an antibody with an invariant or constant region of a human antibody.

A humanised antibody is produced by recombinant methods to combine the complimentarity determining regions of an antibody with both the constant (C) regions and the framework regions from the variable (V) regions of a human antibody.

Chimeric antibodies are recombinant antibodies in which all of the V-regions of a mouse or rat antibody are combined with human antibody C-regions. Humanised antibodies are recombinant hybrid antibodies which fuse the complimentarily determining regions from a rodent antibody V-region with the framework regions from the human antibody V-regions. The C-regions from the human antibody are also used. The complimentarily determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Preferably said fragments are single chain antibody variable regions (scFV's) or “domain” antibody fragments. If a hybidoma exists for a specific monoclonal antibody it is well within the knowledge of the skilled person to isolate scFv's from mRNA extracted from said hybridoma via RT PCR Alternatively, phage display screening can be undertaken to identify clones expressing scFv's. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety.

In a preferred embodiment of the invention said fragment is a Fab fragment.

In a further preferred embodiment of the invention said antibody is selected from the group consisting of: F(ab′)₂, Fab, Fv and Fd fragments; CDR3 regions; single chain variable region fragments; or domain region fragments.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not illicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In a further preferred embodiment of the invention said antibodies are opsonic antibodies.

Phagocytosis is mediated by macrophages and polymorphic leukocytes and involves the ingestion and digestion of micro-organisms, damaged or dead cells, cell debris, insoluble particles and activated clotting factors. Opsonins are agents which facilitate the phagocytosis of the above foreign bodies. Opsonic antibodies are therefore antibodies which provide the same function. Examples of opsonins are the Fc portion of an antibody or compliment C3.

In a further aspect of the invention there is provided a method for preparing a hybridoma cell-line producing monoclonal antibodies according to the invention comprising the steps of:
i) immunising an immunocompetent mammal with a conjugate, composition, nucleic acid or vector according to the invention;
ii) fusing lymphocytes of the immunised immunocompetent mammal with myeloma cells to form hybridoma cells;
iii) screening monoclonal antibodies produced by the hybridoma cells of step (ii) for binding activity to the antigen of the conjugate according to the invention;
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

According to a further aspect of the invention there is provided a hybridoma cell-line obtainable by the method according to the invention.

An embodiment of the invention will now be described by example only and with reference to the following materials, methods and sequences.

Materials and Methods

1. Production of an Antigen-Adjuvant Conjugate

a. Production of Recombinant Adjuvant Protein.

The VI domain (a repeating unit) of DiAg gene was amplified by polymerase chain reaction (PCR) with primers (5_-primer, including NdeI restriction site: 5_-GCATATGAATGAT-CATAATTTAGAAAGC-3_{—},3_-primer, including BamHI restriction site: 5_-CTAAAGGATCCTATCACCGCTTACGCCGTTCATTCATTG-3_) from from a D.immitis cDNA library. Amplified DNA was digested with NdeI and BamHI and cloned into pET3a vector (Stratagene) for expression in E. coli HMS174 (DE3). The purification of rDiAg was performed as follows. Five g of cell paste was suspended in 30 ml of 50 mM HCl and 5 mM EDTA at 4° C. and then centrifuged at 12,000 g for 10 min. Recombinant DiAg in the supernatant was precipitated by 60-80% saturated ammonium sulfate and then applied on a Superdex 200 column. Contaminants of pyrogen from E. coli were removed from concentrated rDiAg solution by immobilized polymixin B. The isolated adjuvant protein was lyophilized and stored at −20° C. until use.

b. Conjugation of Adjuvant Protein to Antigen

One of a number of possible methods for conjugating a peptide to an antibody would be as follows, by way of example only:

The D.immitis polyladder protein V1 domain adjuvant (YFQTYLSWLTDAQKDEIKKMKEEGKSKM I QKKI F D Y F ES LTGDKKKKAAEELQQGCLMALSEIIGNEKMLMLKEIKDSGADPEQIEDMLKLVVDKEKK KRIDEYPPVCRKIYAAMNERRK) (Adjuvant) is dissolved in 0.1M Sodium phosphate, 0.15M NaCl, pH 7.2 at a concentration of 3-30 mg/ml. 6 mg of sulfo-SMCC (Pierce) are added, and the mixture incubated at room temperature for 30 min. Excess cross-linker is then immediately removed on a desalting column (Sephadex G-25) or by ultrafiltration using 0.1M Sodium phosphate, 0.15M NaCl, pH 7.2 as the chromatography buffer. Fractions containing the peptide (by OD280) are pooled and the maleimide activated peptide concentrated to around 10 mg/ml.

In the meantime, antigen, for example, purified recombinant HSV glycoprotein D antigen (gD) is dissolved in or exchanged into the 0.1M Sodium phosphate, 0.15M NaCl, pH 7.2 buffer at 1-5 mg/ml. Add 10-40 μl of SATA (Pierce) stock solution (8 mg/ml in DMSO or DMF) for each ml of gD at 1 mg/ml. React for 30 min at room temperature. gD is then purified away from unreacted SATA by dialysis, gel filtration or ultrafiltration.

Acetylated sulphydryl groups on the SATA modified gD are then de-protected as follows:

A 0.5M hydroxylamine solution in 0.1M sodium phosphate, ph7.2 with 10 mM EDTA is prepared. 100 ul of this solution is added to each ml of antibody and left for 2h at room temperature. The thiolated gD is then purified by utrafiltration into 0.1M sodium phosphate, 0.1M NaCl, pH 7.2, 10 mMEDTA, and immediately mixed with maleimide activated DiAg at a 1:10 molar ratio of gD to DiAg. The reaction is allowed to continue for two hours at 37C. Conjugated gD-DiAg is then purified away from unreacted DiAg by ultrafiltration.

2. Production of an Antigen-Adjuvant Fusion Protein

The V1 domain (a repeating unit) of the Dirofilaria immitis polyladder protein was amplified by polymerase chain reaction (PCR) with primers (5_-primer, including HindII restriction site: 5_GAAGCTTAATGATCATAAGGGAGAAAGC-3_{—},3_-primer, including BamHI restriction site: 5_-CTAAAGGATCCTATCACCGCTTACGCCGTTCATTCATTG-3_) from a D.immitis cDNA library. Amplified DNA was digested with Hind III and BamHI.

Meanwhile gD encoding DNA was amplified from HSV infected cells using primers incorporating additional nucleic acids and a Bam H1 restriction site in the 5′ primer such that upon digestion with Bam H1 and ligation to the DiAg encoding fragment, the DiAg and the gD encoding cDNAs were in-frame with each other, allowing continuous transcription of the DNA into an mRNA translatable into a fusion protein consisting of DiAg and gD. These two DNA fragments were ligated into a mammalian expression vector (pcDNA3.1.(InVitrogen)) and this plasmid was used to transform competent E.coli cells allowing the production of sufficient quantity of plasmid (purified using a Qiagen column) to transfect COS-7 cells by electroporation. Fusion protein was purified from transfected COS-7 cell supernatant after 3-5 days using an anti-DiAg affinity column, and after further purification by gel filtration, fusion protein was used for immunisation.

3. Production of a DNA Vaccine Encoding an Antigen-Adjuvant Fusion Protein.

The pcDNA3.1 expression vector with insert, produced as described above, was used directly as a DNA vaccine by intramuscular injection

### REFERENCES

Tomlinson et al. J. Immunol. 143 2349-2356 (1989);Paxton et al. Infect Immunol. 61 2827-2833 (1993)

We claim:

1-3. (canceled)

4. A composition comprising at least one polypeptide wherein said polypeptide comprises;
an amino acid motif consisting of the amino acid residues
RXK/RR wherein R is arginine, X is any amino acid residue, and K is lysine; and/or
an amino motif consisting of the amino acid residues
RXFR wherein F is phenylalanine and further wherein said motif(s) is preceded by a cysteine amino acid residue about 7-9 residues amino terminal to said motif(s) which polypeptide can be modified by addition, deletion, or substitution of at least one amino acid residue; and
iii) at least one antigen to which an immune response is desired.

5. A composition according to claim 4 wherein said polypeptide is mixed with said antigen.

6. A composition according to claim 4 wherein said polypeptide is conjugated, associated or crosslinked to said antigen.

7. A composition according to claim 4 wherein said polypeptide comprises an amino acid sequence set forth as SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO:8; SEQ ID NO:9; SEQ ID NO:10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; or a polypeptide which is at least 50% homologous to said polypeptides.

8. A composition according to claim 4 wherein said polypeptide comprises an amino acid sequence as set forth as SEQ ID NO. 1: SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; or a polypeptide which is at least 70% homologous to said polypeptides.

9. A composition according to claim 4 wherein said polypeptide comprises an amino acid sequence as set forth as SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; or a polypeptide which is at least 90% homologous to said polypeptides.

10. A composition according to claim 4 wherein the length of said polypeptide is at least 20 consecutive amino acids identical in sequence to at least a 20 amino acid portion of a sequence as set forth as SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; or SEQ ID NO: 19.

11. A composition according to claim 4 wherein said polypeptide is a lymphocyte binding fragment of such a protein.

12. A composition according to claim 4 wherein said polypeptide is encoded by a nucleic acid molecule comprising a nucleic acid sequence set forth as SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; or SEQ ID NO: 34; or a nucleic acid molecule which hybridises to said nucleic acid molecule under stringent hybridization conditions and which encodes a polypeptide with immunological adjuvant activity.

13. (canceled)

14. A composition according to claim 12 wherein said polypeptide is encoded by a 60 nucleotide portion of a nucleic acid sequence set forth as SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32, SEQ ID NO: 33; or SEQ ID NO: 34.

15. (canceled)

16. A composition according to claim 4 wherein said polypeptide is produced as fusion protein with said antigen.

17. A composition according to claim 4 wherein said polypeptide and said antigen are encapsulated in synthetic microparticles or nanoparticles, liposomes, or immune stimulating complexes.

18. A composition according to claim 4 wherein said polypeptide and said antigen are co-adsorbed or co-precipitated onto aluminium.

19. A composition according to claim 4 wherein said polypeptide and said antigen are co-adsorbed or co-precipitated onto calcium salts.

20. A composition according to claim 4 wherein said polypeptide and said antigen are encoded by the same nucleic acid molecule.

21. A composition according to claim 4 wherein said polypeptide and said antigen are linked as an in frame fusion.

22. A composition according to claim 21 wherein in frame fusion includes a linker nucleic acid molecule encoding a flexible linker sequence.

23. A composition according to claim 21 wherein said composition comprises at least two copies of said polypeptide fused to one copy of said antigen.

24. (canceled)

25. A composition according to claim 21 wherein said composition comprises a single copy of said polypeptide fused to at least two copies of said antigen.

26. (canceled)

27. A composition according to claim 21 wherein said polypeptide is a Dirofilaria polypeptide.

28. A composition according to claim 27 wherein said polypeptide is a Dirofilaria immitis polypeptide.

29. A composition according to claim 21 wherein said composition further comprises a carrier.

30. A composition according to claim 4 wherein said composition comprises a second adjuvant.

31. A composition according to claim 4 wherein said antigen is a T-cell dependent antigen.

32. A composition according to claim 4 wherein said antigen is a T-cell independent antigen.

33. A composition according to claim 4 wherein said antigen is from a pathogenic bacterium.

34. A composition according to claim 33 wherein said bacterial species comprises Staphylococcus aureus; Staphylococcus epidermidis; Enterococcus faecalis; Mycobacterium tuberculsis; Streptococcus group B; Streptoccocus pneumoniae; Helicobacter pylori; Neisseria gonorrhoea; Streptococcus group A; Borrelia burgdorferi; Coccidiodes immitis; Histoplasma sapsulatum; Neisseria meningitidis type B; Shigella flexneri; Escherichia coli; Haemophilus influenzae, Chalmydia trachomatis, Chlamydia pneumoniae, Chlamydia psittaci, Francisella tularensis, or Bacillus anthracis.

35. A composition according to claim 4 wherein said antigen is from a viral pathogen.

36. A composition according to claim 35 wherein said viral pathogen comprises Human Immunodeficiency Virus (HIV1 and 2); Human T Cell Leukaemia Virus (HTL 1 and 2); Ebola virus; human papilloma virus (HPV); papovavirus; rhinovirus; poliovirus; herpesvirus; adenovirus; Epstein Barr virus; influenza virus A, B or C, Hepatitis B and C viruses, Variola virus, or rotavirus.

37. A composition according to claim 4 wherein said antigen is from a parasitic pathogen.

38. A composition according to claim 37 wherein said parasitic pathogen comprises Trypanosoma cruzi, Trypansosoma brucei, Schistosoma spp; Plasmodium spp. Loa Loa, Leishmania spp; Ascaris lumbricoides, Dirofilaria immitis, or Toxoplasma gondii.

39. A composition according to claim 4 wherein said antigen is derived from a fungal pathogen.

40. A composition according to claim 39 wherein said fungal pathogen is of the genus Candida.

41. A composition according to claim 4 wherein said antigen is a tumour specific antigen.

42. A composition according to claim 41 wherein said antigen is a hormone involved in hormone dependent cancer.

43. A composition according to claim 4 wherein said antigen is a human host antigen.

44. A composition according to claim 43 wherein said human host antigen is a hormone; hormone receptor; T cell receptor; or sperm antigen.

45. A composition according to claim 4 wherein said antigen is a prion protein.

46. A composition according to claim 45 wherein said antigen is an amyloid protein or a fragment thereof.

47. A composition according to claim 46 wherein said fragment comprises the amino acid sequence, DAEFRHDSGYEVHHQKLVFFADEVGSNKGAIIGLMVGGVVIA, or variant thereof.

48-66. (canceled)
